(19) United States
(12) Patent Application Publication
(10) Pub. No.: US 2007/0265615 A1
(43) Pub. Date: Nov. 15, 2007

(54) Electrosurgical instrument with suction, irrigation and means to collect blood

(76) Inventor: Haim Ben-Simhon, Haifa (IL)

Correspondence Address: Dr. HAIM BEN-SIMHON, 18/24 DREIFUS STREET, HAIFA, 35434, omitted

(21) Appl. No.: 11/473,103

(22) Filed: Jun. 23, 2006

(30) Foreign Application Priority Data
May 9, 2006 (IL) 175493

(51) Int. Cl. A61B 18/14

(52) U.S. Cl. 606/45; 606/49

(57) ABSTRACT

An electrosurgical instrument with electrode, suction, optional irrigation and means to bend the distal part of the surrounding suction tube is described. A distal part of the suction tube (around the electrode) has two members, whereas one member can be engaged inside the other—to various degrees—in order to control the length of the suction tube and its relation to the tip of the electrode. Means to fixate the electrode within the suction tube are described. Also described are means to evacuate smoke, blood, irrigation anticoagulant fluid and debris via a suction tube to a separation chamber and evacuation of the gaseous portions by a vacuum machine. The separation chamber serves as a blood collection container (the blood could then be re-infused to the patient!). Irrigation means of isotonic fluid with blood-anticoagulant combined with the suction are described. Combination of the various functions and options could be utilized for building various useful surgical instruments.

### BACKGROUND OF THE INVENTION

Electrosurgical instruments with suction and irrigation properties has been described; However, means to enable bending of the suction tube alongside with the electrode without kinking of the suction tube has not. Bending of the electrode (usually blade-electrode) is needed when operating on hidden areas, where direct straight approach with the electrode is impossible. Such is the case, when the internal mammary arteries are being developed to be used for coronary bypass operation. The surgeon then bends the electrode to facilitate the approach to the developed arteries.

One purpose of the following invention is to provide the surgeon with an instrument with means to enable bending of the electrode and its surrounding suction tube (without kinking of the tube), thus enabling efficient suction-evacuation of the smoke, blood and debris along the way of the developed structure, and consequently enabling appropriate visualization of the “hidden” (covert) surgical field.

Existing electrosurgical instruments with suction lack the property of versatility in determining the distance between the tip of the electrode and the orifice of the surrounding suction tube. The distance is too large to enable the efficient and simultaneous suction of the blood and coagulation of the cut-open blood vessel (small and medium sizes). A prominent example is, when the implant-pouch for Augmentation Mammoplasty (also known as breast augmentation) is being prepared; Various-size bleeders deep in the pouch have to be coagulated in order to “dry” the pouch before the breast implant could be inserted. To enable the speedy coagulation of these bleeders, the distance between the active tip (portion) of the electrode and the surrounding orifice of the suction tube has to be sufficiently small, or else no simultaneous coagulation and suction could take place. Therefore, other purpose of this invention is to provide an electro-surgical-suction instrument with the characteristics of having means (two in the following description) of changing the size of the distance between the tip of the electrode and the surrounding orifice of the tube at the will of the surgeon.

### Disclosure:

### BRIEF DESCRIPTION OF THE DRAWINGS OF THE INVENTION

FIG. 1: General view of the electrosurgical-suction-irrigation instrument with isotonic fluid (with anticoagulant) supply, a chamber for separation of fluid from gas (air), a vacuum source, the electrosurgical hand-piece, and the two disengaged members of the suction tube surrounding the electrode.

FIG. 2: Screwed (engaged) members of the suction tube surrounding the fixed electrode of the electrosurgical instrument.

FIG. 3: One embodiment of the electrode with wings for fixating the electrode within the distal member of the suction tube.

FIG. 4: Cross-section of the electrode disposed within the suction tube in an eccentric positioning, in relation to the surrounding distal member of the suction tube. The eccentric positioning of the electrode is achieved by means of the asymmetric wings.

FIG. 5: Cross-section of the electrode disposed within the suction tube in a centric positioning, in relation to the surrounding distal member of the suction tube. The central positioning is achieved by means of symmetrical wings extending peripherally from the isolated part of the electrode.

FIG. 5a: Wings extending centrally toward the electrode from the wall of the distal member of the suction tube, close to the orifice of said tube, and said wings unite and merge into a ring around the electrode, whereas the electrode becomes disposed loosely within said ring.

Please refer to the above figures.

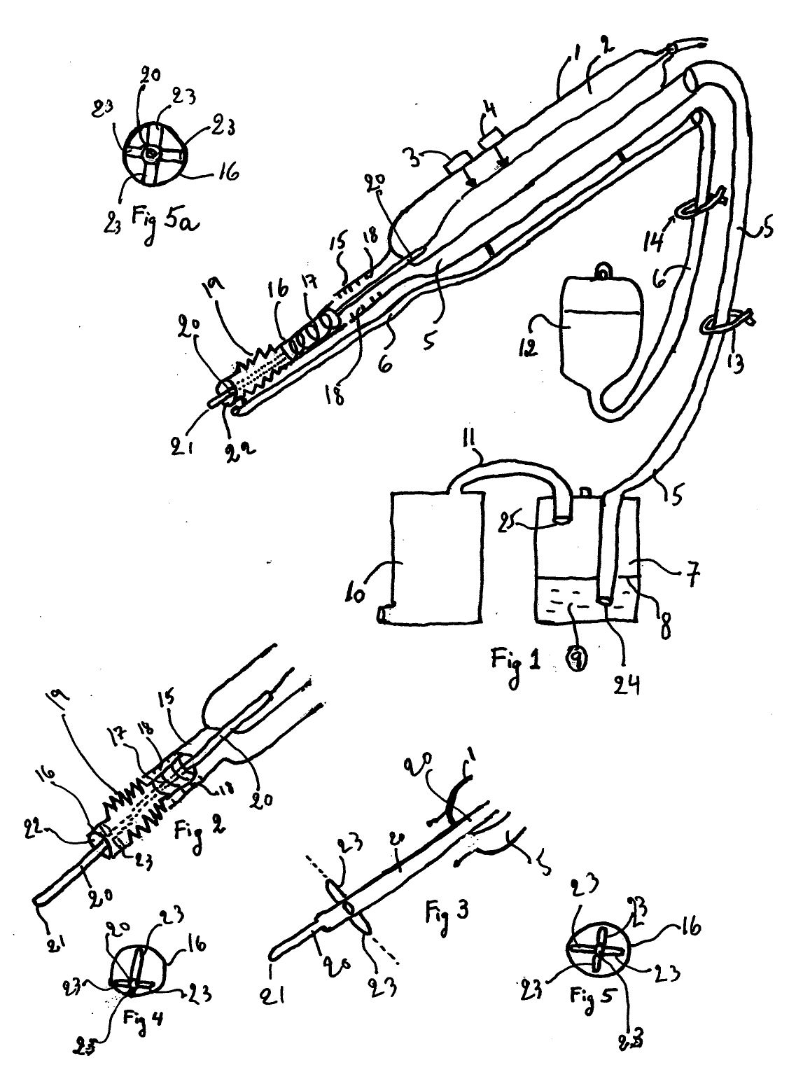

FIG. 1 includes the electrosurgical instrument having a conventional plastic handle 1, electric compartment 2, coagulation knob 3, cutting knob 4, suction tube 5 and irrigation—with isotonic fluid (with anticoagulant)—tube 6. The suction tube 5 is connected to a closed chamber 7 under a fluid level 8. The closed chamber traps blood and debris in its fluid lower part 9, and connects to a vacuum source through its upper (air-filled) part by means of tube 11. This configuration allows the separation of blood and debris from air, which come mixed in suction tube 5. The non-gaseous portion of the mixture sinks to the bottom of the chamber 9, thus protecting the vacuum machine 10 from fluid and keeps it dry.

The blood and the anticoagulant can then be re-infused to the patient (in case of significant amount of bleeding!).

The irrigation tube 6 is connected to the bag of isotonic fluid 12 (isotonic—to preserve the blood cells). Both the suction tube 5 and the irrigation tube 6 have means to control the rate of flow—13 and 14 respectively in FIG. 1. The irrigation tube 6 is attached to the suction tube 5 and to the handle 1 at several points.

The distal part of the suction tube 5 has two members: proximal 15 and distal 16. The distal 16 screws into and engages the proximal part 15 by means of wide spiral screw. The entering distal part is described as the “spiral representation” 17 and the receiving internal screw in the proximal member is represented by the tiny small lines 18 (as it would show if the proximal tube was cut longitudinally—note the relatively large intervals between the small lines representing the receiving wide spiral). The distal tube 16 has a segment 19, which is made of a plastic “accordion” arrangement of the tube (like the bendable portion of a drinking straw). This “accordion” 19 enables to elongate or shorten the distal tube in relation to the electrode 20, thus determining the distance between the tip of the electrode 21 and the orifice 22 of the distal part of the suction tube 16.

The accordion segment 19 could also enable (plastic) to bend the distal part 16 simultaneously with the bending of the electrode 20 without kinking of the suction tube.

The wide spiral screw (17 into 18) also enables to determine the distance between tip 21 of the electrode and orifice 22 of the suction tube. FIG. 1 shows the two members of the suction tube disengaged; they could be screwed partially (small interval or portion only!), thus resulting in a small distance between tip 21 and orifice 22 (as shown in FIG. 1). In FIG. 2 the two members are fully engaged (screwed), resulting in large distance between tip 21 and orifice 22.

The “accordion” structure could also serve to determine the distance between tip 21 and orifice 22 by simply pulling (widening or elongating) or “squeezing” (shortening) of the “accordion” segment 19 of the distal part section 16 of the suction tube.

FIG. 3 describes the electrode 20 (the distal part of the suction tube was removed in this figure for clarity). The electrode has wings 23 attached to it, either asymmetrically as in FIG. 4, or symmetrically as in FIG. 5. The wings in this embodiment are not attached to the suction tube 16, but rather the edge of the wings spans centrifugally close to the wall of the suction tube 16. The wings enable to fixate the electrode in a fixed position within and in relation to the surrounding suction tube.

Another embodiment of this feature is wings attached to the walls of the suction tube and extending from those walls towards the center of the hollow part of the tube, and ending in a ring formation around the electrode—FIG. 5a; thus the electrode is disposed loosely within that ring (FIG. 2 hints this embodiment). These arrangements allow both: sliding of the tube in relation to the electrode, and fixation of the electrode (from moving sideways) within the hollow part (lumen) of the tube.

### Mode of Operation:

The instrument is used to cut tissue and coagulate bleeding blood vessels. The suction is utilized to evacuate smoke, blood and debris. The bag 12 has isotonic fluid with anticoagulant and the rate of flow is controlled by means 14 (in the form of metal clip in this case). By pressing (with the clip) on tube 6 at various degrees of pressure, one gets various degrees (rates) of flow. The fluid with the anticoagulant mixes with the blood near orifice 22, thus preventing the blood from coagulating (clotting) and consequently preserves it for reuse in the patient—(coagulated blood cannot be used for infusion!!).

The suction tube 5 conveys the mixture of anticoagulant fluid, blood, smoke and air to the closed separation-chamber 7. The mixture enters the lower part, which is filled of isotonic fluid, at point 24. The blood and the fluid remain in the bottom, while the gaseous components are sucked through entrance 25, which is disposed in the air part of the chamber (upper part), and are evacuated by the vacuum machine 10 through tube 11. This chamber serves both as a blood collector and gas/fluid separator, and protects the vacuum machine from getting wet by blood or fluid, which are kept in the bottom of the chamber by gravitation.

The mode of activation of the distal parts of the suction tube 15 and 16 is as follows: when the surgeon prefers to have a long part of the electrode 20 exposed out of the suction tube he simply screws part 16 into part 15, and/or squeezes the accordion segment 19 of the distal suction tube 16 (this configuration-position is described in FIG. 2). If the surgeon wants to coagulate cut-open blood vessels and simultaneously suck the blood and debris, then he could “open” the accordion—meaning, having the accordion segment 19 longer, and/or unscrew part 16 from part 15 in the out-direction (without separating completely part 16 from part 15!!); consequently the suction tube would be longer, and the length of the electrode 20, that is exposed (or bulging) out of the tube, would be Shorter (like the case represented in FIG. 1). The surgeon can play with these two characteristics to achieve the optimal positioning for him. The “accordion”-segment-structure also enables bending without kinking of that segment of tube 16 (kinking would block the suction tube!!!—namely no blood or smoke would be evacuated).

The wings of the electrode in FIG. 3, 4, 5 or the wings of the suction tube in FIG. 2 and 5a, enables the fixation of the electrode in relation to the walls of the suction tube, thus preventing the free movement to the sideways, while allowing the longitudinal movement of the distal suction tube relative to the electrode.

Various sizes of distal-tube-part 16 could be used together with various sizes of fitting electrodes for the various needs of various operations (surgical procedures).

1. Electrosurgical instrument with suction and means to bend the suction tube parallel to bending of the electrode without kinking of the suction tube.

2. Electrosurgical instrument having two members of a suction tube with means to engage one member into the other and means to fixate the electrode which is disposed within the said suction tube from moving sideways while allowing longitudinal movement of part of said suction tube relative to the electrode.

3. Electrode of an electrosurgical instrument with wings attached to it in order to fixate the electrode within a suction tube.

4. Claim 1 and said means is an accordion-like plastic segment.

5. Claim 2 and said means are spiral screw for engaging one member of the suction tube into the other.

6. Claim 1 and irrigation means with means to separate suction-evacuated fluid and solids from the suction-evacuated gaseous portions.

7. Claim 1 and a vacuum machine.

8. Claim 1 and means to collect and prevent the coagulation of the evacuated blood.

9. Claim 3 and a suction tube with eccentric positioning of the electrode in relation to the said surrounding suction tube.

10. Claim 3 and a suction tube with central positioning of said electrode in relation to the said surrounding suction tube.

11. Claim 1 whereas the suction tube lays adjacent to irrigation tube which connect to a source of isotonic fluid with anticoagulant; said suction tube connects to collection chamber and to means for separation of fluids from gaseous portions which in turn are evacuated by a vacuum machine.

12. Claim 3 and claim 4.

13. Claim 4 and claim 5.

14. Claim 6 and claim 8.
